(19) United States
(12) Patent Application Publication
(10) Pub. No.: US 2021/0236855 A1
(43) Pub. Date: Aug. 5, 2021

(54) SYSTEMS AND METHODS OF QUALITY ASSURANCE FOR RADIOTHERAPY

(71) Applicants: DUKE UNIVERSITY, Durham, NC (US); THE UNIVERSITY OF BRITISH COLUMBIA, VANCOUVER (CA); BC CANCER (Part of the Provincial Health Services Authority), Kelowna (CA)

(72) Inventors: Justus ADAMSON, Durham, NC (US); Mark OLDHAM, Durham, NC (US); Jaclyn CARROLL, Durham, NC (US); Michael TRAGER, Durham, NC (US); Andrew JIRASEK, Kelowna (CA); Michelle HILTS, Kelowna (CA)

(21) Appl. No.: 17/052,664

(22) PCT Filed: May 3, 2019
(86) PCT No.: PCT/US19/30642

§ 371 Date: Nov. 3, 2020

Related U.S. Application Data
(60) Provisional application No. 62/666,285, filed on May 3, 2018.

(51) Int. Cl. A61N 5/10

(52) U.S. Cl. CPC A61N 5/1075; A61N 5/1045

(57) ABSTRACT

Radiotherapy quality assurance (QA) systems and methods are provided that incorporate a shared frame of reference between a treatment plan and a measured dose distribution that allows for 3D dosimetry measurements. An on-board imaging system may provide a shared frame of reference with the radiotherapy treatment system. A dosimeter is also provided for use with the QA systems and methods. The QA systems and methods can be applied as an end-to-end test to evaluate specific parameters of a radiation therapy treatment system, such as an external beam radiotherapy system, including spatial accuracy, isocenter verification and dosimetric accuracy.

### CROSS-REFERENCE TO RELATED APPLICATIONS

This application is a U.S. national phase of the international patent application No. PCT/US2019/030642 filed on May 3, 2019 and titled “Systems and Methods of Quality Assurance for Radiotherapy,” which claims priority from the U.S. Provisional Patent Application Ser. No. 62/666,285 filed on May 3, 2018 and entitled “Systems and Methods for End-to-End Quality Assurance Test for Radiotherapy.” The disclosure of each of the above identified patent applications is incorporated by reference herein.

### STATEMENT REGARDING FEDERALLY SPONSORED RESEARCH

N/A

### BACKGROUND

Numerous maladies, such as intracranial malignancies, are commonly treated using radiosurgery. For decades, such treatment has been implemented using linear accelerators. Historically, the treatment technique consisted of arcs with conical collimators and more recently has included dynamic conformal arcs (DCA) collimated by High Definition Multi-Leaf Collimators (HDMLC). Multifocal disease has also been treated using a single isocenter volumetric modulated arc therapy (VMAT) treatment plan.

A hallmark of radiosurgery is the highly stringent requirements for spatial localization, with margins on the order of 1 mm. Multifocal VMAT stereotactic radiosurgery (SRS) in particular requires increased attention due to potential rotational errors as small targets may be located at a distance from the isocenter. The spatial accuracy of the linear accelerator radiosurgery system is traditionally verified via the “Winston-Lutz” quality assurance (QA) test, in which the location of a fiducial positioned at isocenter is verified radiographically relative to the beam profile for various treatment geometries. While the Winston-Lutz test was originally designed for cone based radiosurgery, updated procedures have been proposed for MLC based single or and multifocal SRS.

While these QA procedures serve to verify the general spatial stability and accuracy of the radiosurgery system, they are augmented by end-to-end SRS system verification tests and, in the case of multifocal VMAT, patient specific quality assurance tests. Comprehensive end to end tests to verify geometric accuracy are recommended in guidance documents, and typically consist of hidden targets placed in a head phantom with verification by portal imaging. In addition to spatial accuracy, dose may also be verified; this is most commonly accomplished using radiochromic film. However for multifocal VMAT one challenge is the difficulty in capturing dosimetric information for all targets with a single measurement, especially at an appropriately high resolution.

3D dosimetry systems may have unique advantages for these special circumstances, in that they can offer comprehensive dosimetric measurements at very high resolution. Traditionally, radiation dose measurements are made by measuring a change in the dosimeter material that is manifest as a change in optical density and is read out using optical CT, and/or a change in MRI signal. Despite the potential advantages of comprehensive and high resolution dosimetry, 3D dosimetry is not routinely used with multifocal SRS, likely because they require a dose calibration, and are challenged by the limited access to optical CT or MRI for reading out dose information, and lack of specialized commercial analysis, thus making it impractical for routine clinical application. In addition, because the dose is reconstructed using an independent system (typically optical CT or MRI), the planned and measured dose matrices must be registered in the analysis software; this adds another uncertainty to the analysis and spatial accuracy.

### SUMMARY OF THE DISCLOSURE

The present disclosure addresses the aforementioned drawbacks by providing systems and methods for quality assurance in radiotherapy that includes a shared frame of reference between the treatment plan and the measured dose distribution and allows for 3D dosimetry measurements.

In one configuration, a method is provided for quantifying the spatial accuracy, mechanical parameters, and/or output of a radiation therapy system by performing a quality assurance (QA) test. The method includes positioning a dosimeter containing a polymer gel material in the radiation therapy system. The polymer gel material has a material property that changes in response to therapeutic radiation, which is measurable by an imaging modality. The method also includes acquiring a pre-irradiation volumetric image of the dosimeter. The method also includes irradiating the dosimeter with therapeutic radiation using the radiation therapy system to change the material property of the dosimeter and create a region of increased contrast in the polymer gel material of the dosimeter. A post-irradiation volumetric image may be acquired of the dosimeter to image the region of increased contrast. A QA report may then be generated based upon the post-irradiation image with the region of increased contrast and the pre-irradiation image.

In one configuration, a system is provided for quantifying the spatial accuracy, mechanical parameters, and/or output of a radiation therapy system by performing a quality assurance (QA) test. The system includes a dosimeter containing a polymer gel material with a material property that changes in response to therapeutic radiation, which is positioned in the radiation therapy system. The radiation therapy system is configured to irradiate the dosimeter with therapeutic radiation to change the material property of the dosimeter and create a region of increased contrast in the polymer gel material of the dosimeter. The system also includes an imaging system configured to: i) acquire a pre-irradiation volumetric image of the dosimeter prior to irradiating the dosimeter; and ii) acquire a post-irradiation volumetric image of the dosimeter to image the region of increased contrast. The system also includes a computer system configured to generate a QA report based upon the post-irradiation image with the region of increased contrast and the pre-irradiation image.

In one configuration a kit is provided for performing quality assurance (QA) testing of a radiation therapy system. The kit includes a dosimeter containing a polymer gel material with a material property that changes in response to therapeutic radiation in a radiation therapy system. The change in the material property is quantifiable as a region of increased contrast in a post-irradiation volumetric image of the dosimeter acquired using an imaging system. The kit also includes a computer readable medium configured to access the post-irradiation volumetric image of the dosimeter. The computer readable medium contains instructions stored on the computer readable medium for identifying the region of increased contrast and generating a QA report based upon the post-irradiation image with the region of increased contrast.

In one configuration, a computer readable medium is provided. The computer readable medium includes instructions stored on the computer readable medium for accessing a pre-irradiation volumetric image of a dosimeter containing a polymer gel material with a material property that changes in response to therapeutic radiation in a radiation therapy system. The change in the material property is quantifiable as a region of increased contrast in a post-irradiation volumetric image of the dosimeter acquired using an imaging system. The computer readable medium also includes instructions for accessing a post-irradiation volumetric image of the dosimeter, where the post-irradiation volumetric image includes the region of increased contrast created by therapeutic radiation that has changed the material property of the dosimeter. The computer readable medium also contains instructions for generating a QA report based upon the post-irradiation image with the region of increased contrast and the pre-irradiation image.

In one configuration, a dosimeter is provided for use in a radiation therapy system. The dosimeter includes a polymer gel including an oxygen scavenger material and a material with a material property that changes in response to therapeutic radiation in a radiation therapy system, where the change in the material property is quantifiable using an imaging system.

In some configurations, the polymer gel material includes 3-20 wt % of N-isopropylacrylamide (NIPAM). In some configurations, the polymer gel material includes 3-7 wt % of N,N′-methylenebisacrylamide (bis). In some configurations, the polymer gel material includes 2-10 wt % of gelatin.

In some configurations, the oxygen scavenger material includes at least one of tetrakis hydroxymethyl phosphonium chloride (THPC), ascorbic acid, copper sulfate, gallic acid, Trolox, or N-acetyl-cysteine. In some configurations, the oxygen scavenger material is THPC and the dosimeter includes 5 mM-100 mM of THPC.

In some configurations, the dosimeter also includes de-ionized water. In some configurations, the dosimeter comprises 63-92 wt % of de-ionized water.

In some configurations, 5 mM-100 mM of THPC is used for remote dosimetry applications. In other configurations, 10 mM-100 mM of THPC is used for remote dosimetry applications.

The foregoing and other aspects and advantages of the present disclosure will appear from the following description. In the description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention.

### BRIEF DESCRIPTION OF THE DRAWINGS

FIG. 1 is a block diagram of a radiation therapy system that may be used in accordance with the present disclosure.

FIG. 2A is an image of a non-limiting example dosimeter.

FIG. 2B is a cross sectional image of the dosimeter of FIG. 2A using an imaging system in accordance with the present disclosure.

FIG. 3A is a flowchart of non-limiting example steps for a method of performing isocenter verification of a radiation therapy system in accordance with the present disclosure.

FIG. 3B is a flowchart of non-limiting example steps for a method of performing QA for a radiation therapy system in accordance with the present disclosure.

FIG. 4A a flow chart of non-limiting example steps for a QA end-to-end test of spatial accuracy in accordance with the present disclosure.

FIG. 4B is another flow chart of non-limiting example steps for a QA end-to-end test of spatial accuracy in accordance with the present disclosure

FIG. 5A is a graph of non-limiting example results where the absolute volume of structures are graphed as a function of the threshold value for regions of interest surrounding each target.

FIG. 5B is a graph of non-limiting example results where the volumes shown in FIG. 5A are normalized to the volume of the prescription dose per target from the treatment plan.

FIG. 5C is a graph of non-limiting example results where a similar analysis to that depicted in FIGS. 5A and 5B is used, in which spatial location is also included.

FIG. 6 is a flow chart depicting non-limiting example steps for a method for verification of dosimetric accuracy for multi-target radiosurgery in accordance with the present disclosure.

FIG. 7 a non-limiting example dosimeter 700 is shown that may be used for both dose calibration and to test irradiation.

FIG. 8 non-limiting example results are shown for the planned and measured dose distribution using the dosimeter of FIG. 7.

### DETAILED DESCRIPTION

Before the present invention is described in further detail, it is to be understood that the invention is not limited to the particular embodiments described. It is also understood that the terminology used herein is for the purpose of describing particular embodiments only, and is not intended to be limiting. The scope of the present invention will be limited only by the claims. As used herein, the singular forms “a”, “an”, and “the” include plural embodiments unless the context clearly dictates otherwise.

Specific structures, devices, and methods relating to x-ray imaging are disclosed. It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. When two or more ranges for a particular value are recited, this disclosure contemplates all combinations of the upper and lower bounds of those ranges that are not explicitly recited. For example, recitation of a value of between 1 and 10 or between 2 and 9 also contemplates a value of between 1 and 9 or between 2 and 10.

Radiotherapy quality assurance (QA) systems and methods are provided that incorporate a shared frame of reference between the treatment plan and the measured dose distribution and allows for 3D dosimetry measurements. A shared frame of reference may be provided by use of an imaging system on-board with the radiotherapy treatment system. In some configurations, a shared frame of reference between the treatment plan and the measured dose distribution may eliminate the need for spatial registration between the treatment system and the imaging system. A three-dimensional (3D) dose measurement at high spatial resolution may also be used. The QA technique can be applied as an end-to-end test to evaluate specific parameters of a treatment system, such as an external beam radiotherapy system, and the like.

In some configurations, the quality assurance systems and methods include capability for remote dosimetry. Remote dosimetry may include considerations for preparation of a dosimeter at a remote location and transportation of the dosimeter to a clinical facility.

In some configurations, minimal measurements and analysis may be used as part of the quality assurance process. The systems and/or methods may provide for ease of analysis which can optionally be performed within the clinical treatment planning system, thus dispensing with a need for external systems and procedures.

The methods of the present disclosure can be applicable to radiosurgery and SBRT, both of which are special cases of radiotherapy. However, it will be appreciated by one skilled in the art that the methods can be applied generally to any external beam radiotherapy technique. The systems and methods may provide for the ability to directly measure essential parameters of an external beam radiotherapy system, such as uncertainty/wobble of the radiation isocenter over the range of potential geometries of the accelerator, coincidence of the imaging and radiation coordinate systems, mechanical accuracy of accelerator geometrical parameters (couch, gantry angle, for examples), and the like. In one non-limiting example, treatment plans that are representative of clinical cases can be prepared and utilized for end-to-end verification of either spatial or dosimetric accuracy.

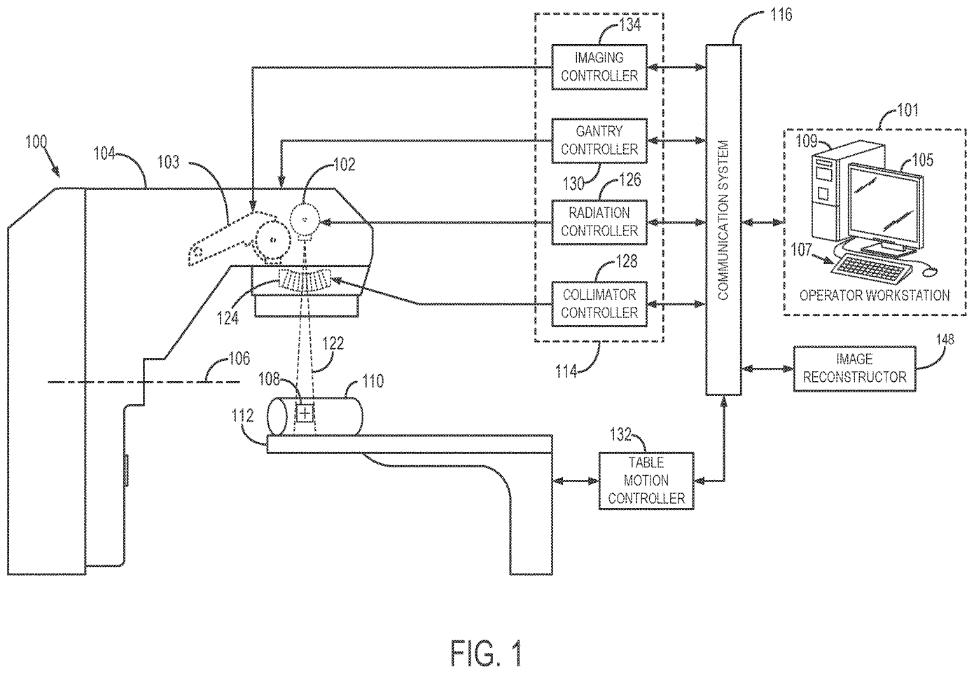

Referring to FIG. 1, an exemplary radiation therapy system 100 includes a therapeutic radiation source 102 and an on-board imaging source 103. The radiation source 102 and the on-board imaging source 103 may be housed in the same gantry system 104 or may be mounted orthogonally to the radiation source 102. The radiation therapy system 100 may include any suitable radiation treatment system, including image-guided radiation therapy (“IGRT”) systems, intensity-modulated radiation therapy (“IMRT”) systems such as intensity-modulated arc therapy (“IMAT”) and volumetric modulated arc therapy (“VMAT”) systems, an external beam radiotherapy delivery system, such as a linear accelerator (LINAC), proton radiotherapy systems, slice by slice photon radiotherapy systems (Tomotherapy), non-isocentric photon radiotherapy systems (Cyberknife), and isotope based radiotherapy systems (ViewRay and GammaKnife), and the like. In a non-limiting example, the radiation therapy system is a Truebeam STX linear accelerator with 6 MV photons and HD-Multileaf Collimators (MLC). The treatment beam for the radiation therapy system can be composed of photons, neutrons, electrons, protons, heavy charged particles, or the like. Specific treatment plans can also be designed and delivered in order to evaluate key parameters of each radiotherapy system. Clinically relevant treatment plans can be prepared and utilized for end-to-end testing.

The on-board imaging source 103 may include an x-ray source, a Cone-Beam Computed Tomography (CBCT) system, a Computed Tomography (CT) system, a 4DCT system, a magnetic resonance imaging (MRI) system, and the like. Alternatively, the imaging may be performed by a separate diagnostic imaging system. Both the therapeutic radiation source 102 and imaging source 103 are attached adjacent each other and housed at the same end of a rotatable gantry 104, which rotates about a pivot axis 106. The rotatable gantry 104 allows either of the sources, 102 and 103, to be aligned in a desired manner with respect to a target volume 108 in an object 110 positioned on a table 112.

The rotation of the rotatable gantry 104, the position of table 112, and the operation of the sources, 102 and 103, are governed by a control mechanism 114 of the radiation therapy system 100. The control mechanism 114 includes a radiation controller 126 that provides power and timing signals to the radiation source 102, an imaging controller 134 that provides image acquisition instructions to imaging source 103, and receives image data therefrom, and a gantry motor controller 130 that controls the rotational speed and position of the gantry 104. The control mechanism 114 communicates with an operator workstation 101 and other parts of a network through communication system 116. An image reconstructor 148, receives sampled and digitized image data from the communication system 116 and performs high speed image reconstruction. The reconstructed image is applied as an input to a computer 109.

The computer 109 also receives commands and scanning parameters from an operator via a console that has a keyboard 107. An associated display 105 allows the operator to observe the reconstructed image and other data from the computer 109. The operator supplied commands and parameters are used by the computer 109 to provide control signals and information to the imaging controller 134, the radiation controller 126 and the gantry motor controller 130. In addition, the computer 109 operates a table motor controller 132 which controls the motorized table 112 to position the object 110 within the gantry 104.

Still referring now to FIG. 1, radiation source 102 produces a radiation beam, or “field,” 122, which in some forms may be conical or any other shape, emanating from a focal spot and directed toward an object 110. The radiation beam 122 may be initially conical and is collimated by a collimator 124 constructed of a set of rectangular shutter system blades to form a generally planar “fan” radiation beam 122 centered about a radiation fan beam plane. Each leaf of the collimator is constructed of a dense radio-opaque material such as lead, tungsten, cerium, tantalum, or related alloy.

A collimator control system 128 directed by a timer generating desired position signals provides electrical excitation to each electromagnet to control, separately, actuators to move each of the leaves in and out of its corresponding sleeve and ray. The collimator control system 128 moves the leaves of the collimator 124 rapidly between their open and closed states to either fully attenuate or provide no attenuation to each ray. Gradations in the fluence of each ray, as needed for the fluence profile, are obtained by adjusting the relative duration during which each leaf is in the closed position compared to the relative duration during which each leaf is in the open position for each gantry angle. Alternatively, a physical cone or other structure may be used in place of the multi-leaf collimator.

The ratio between the closed and open states or the “duty cycle” for each leaf affects the total energy passed by a given leaf at each gantry angle, θ, and thus controls the average fluence of each ray. The ability to control the average fluence at each gantry angle, θ, permits accurate control of the dose provided by the radiation beam 122 through the irradiated volume of the object 110 by therapy planning methods to be described below. The collimator control system 128 also connects with a computer to allow program control of the collimator 124 to be described.

An image reconstructor 148, typically including a high speed array processor or the like, receives the data from the imaging controller 134 in order to assist in “reconstructing” an image from such acquired image data according to methods well known in the art. The image reconstructor 148 may also use post-radiation detector signals from a radiation detector to produce a tomographic absorption image to be used for verification and future therapy planning purposes as described in more detail below.

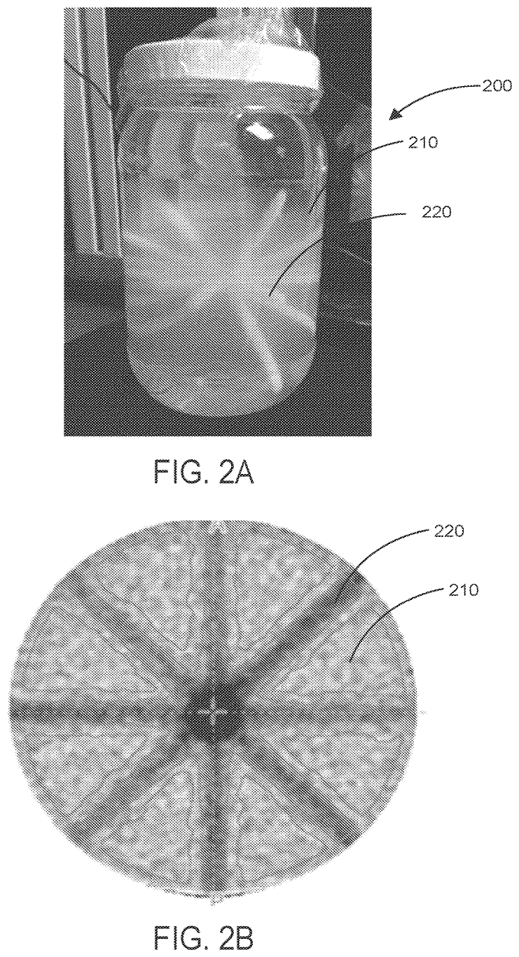

Referring to FIG. 2A, object 110 as shown in FIG. 1 may include a dosimeter 200, for which the radiation dose invokes a change in the density of the dosimeter material 210 which appears as a change in contrast in the volumetric image of the dosimeter material 210, to higher contrast material 220. In some configurations, the dosimeter material 210 is a polymer gel material having a material property that changes in response to therapeutic radiation, the material property being measurable by an imaging modality. The material property may be density. The material property changes, which may present as changes in contrast, may be mapped using the imaging modality to verify the therapeutic radiation delivered to the dosimeter or to verify a parameter of the therapeutic radiation delivered to the dosimeter as part of a QA procedure.

In some configurations, the dosimeter includes an N-isopropylacrylamide (NIPAM)-based polymer gel for which dose invokes a change in density. The changes in density may manifest as changes in intensity or contrast in volumetric images, such as kV-CBCT images acquired using a kV imaging system mounted on board a medical linear accelerator.

In one non-limiting example, the dosimeter gel formulation includes 15% (by weight) N-isopropylacrylamide (NIPAM), 4.5% N,N′-methylenebisacrylamide (bis), 5% gelatin, 75.5% de-ionized water, and 5 mM tetrakis hydroxymethyl phosphonium chloride (THPC, as antioxidant).

In some configurations, the systems and methods of the present disclosure allow for remote dosimetry. While dosimeters can be prepared on-site, they may also be prepared remotely and shipped to a facility performing the end-to-end test. A number of techniques may assist in enabling transportation to a separate, geographically distant facility for the end-to-end QA. These may include cold-packing, adding oxygen absorber materials within the packed volume (since polymer gels are typically oxygen sensitive), adding a layer of liquid such as mineral oil above the gel to eliminate air within the container, or to create a barrier between air and the gel, using specialized containers and packaging, and the like. The choice of dosimeter material and chemical formula may be made so as to facilitate remote dosimetry, such as not requiring refrigeration, being less susceptible to oxygen contamination, and being more long-lived.

In some configurations for remote dosimetry considerations, where the dosimeter may be manufactured at one facility and shipped to a secondary location, THPC concentration may be increased to 10 mM. One skilled in the art will appreciate that a range of concentrations can be used for NIPAM-based polymer gel dosimetry. Non-limiting example ranges for dosimeter gel formulation include:
NIPAM: 3%-˜20% (by weight). Higher amounts of NIPAM may lead to a more sensitive dosimeter. In some configurations, a user may select the concentration of NIPAM based upon a desired sensitivity level.
bis: 3%-7% (higher ranges may be limited by solubility of bis in solution). As with NIPAM, higher bis concentration may lead to a more sensitive dosimeter. One skilled in the art will also appreciate that the relative ratios of NIPAM and bis may play a role in the final sensitivity. In some configurations, a user may select the concentration of bis, or the ratio of NIPAM to bis, based upon a desired sensitivity level.
gelatin: 2%-10%. Lower ranges make a less rigid dosimeter, whereas higher ranges result in a stiff dosimeter, which may be more amenable to remote applications. In some configurations, a user may select the concentration of gelatin based upon a desired stiffness level, or based upon shipping time or range, of the dosimeter.
THPC: ˜5 mM-100 mM. The lower limit may be set by a necessary amount of antioxidant needed to scavenge a desired level of oxygen within the gel. For remote considerations, higher concentrations of THPC may be used due to degradation expected over the time or distance of shipping. However, as THPC concentration ([THPC]) increases, gel sensitivity to radiation dose may decrease. In some configurations, a user may select the concentration of THPC based upon a desired level of oxygen scavenging, or a desired sensitivity level, or based upon shipping time or range.

In some configurations, the dosimeter may be a polymer gel dosimeter. A number of polymer gel dosimeter chemical compositions exist in the literature. Some non-limiting examples include:
PAG: acrylamide, bis, gelatin, water, with or without antioxidant
MAGIC: methacrylic acid-based dosimeters
dosimeters where gelatin is replaced by agarose or gellan gum
Dosimeters with a range of antioxidants, for example, THPC, ascorbic acid, copper sulfate, gallic acid, Trolox, N-acetyl-cysteine etc. These can be used for PAG, NIPAM etc based dosimeters.

In some configurations, using the dosimeters such as those listed above in a QA method according to the present disclosure may lead to measurable changes in a number of measurable parameters, for example, density (for CT), R2 relaxation rate (for MRI).

The formulation process for creating a dosimeter may include controlling for timing and temperature, to ensure reproducibility. In one non-limiting example of a formulation for creating a dosimeter according to the present disclosure, a dosimeter included by weight (1 g=1 ml for pure water of density 1 g/ml): 75.5% deionized water, 5% gelatin (Sigma-Aldrich, Oakville, ON, Canada), 15% N-isopropylacrylamide (NIPAM, TCI Chemicals), 4.5% N,N′-methylenebisacrylamide (BIS, Sigma-Aldrich), and 5 mM tetrakis hydroxymethyl phosphonium chloride (THPC, Sigma-Aldrich). Gelatin (300 Bloom Type A, Sigma-Aldrich) was allowed to swell in the 75.5% of the de-ionized water for 10 min at room temperature, before heating to 45° C. While stirring continuously, Bis was dissolved at 45° C., which took about 15 min, followed by addition of monomer (NIPAM). The gelatin-crosslinker mixture was allowed to cool to approximately 37° C. A solution of the antioxidant THPC was added to the solution. The resulting gels were clear (the Bis gel was very faint yellow) and transparent. The gel solutions were transferred into a plastic container of ˜1 L and with low oxygen permeability, and then closed with a sealing film.

For some 3D dosimeter materials, such as polyacrylamide gels, radiation dose invokes a change in physical density, which is manifest as a change in Hounsfield Unit in a diagnostic x-ray system, such as a CT scanner. Similarly, N-isopropylacrylamide (NIPAM) is a 3D polymer gel material that may be a less toxic alternative to polyacrylamide gel. These materials may be used with diagnostic x-ray systems, such as CT, to reconstruct radiotherapy dose distributions.

Referring to FIG. 2B, a non-limiting example cross section of the dosimeter 200 is shown where the radiation dose changed the contrast of the volumetric image of the dosimeter material 210 to higher contrast material 220.

In some configurations, the dosimeter may be used for isocenter verification. The Winston-Lutz (WL) test was originally designed for cone based SRS in the pre-image guidance era. Using a dosimeter as described above in a method in accordance with the present disclosure allows for a fast & comprehensive method to verify radiation isocenter wander over various gantry and couch angles similar to the Winston-Lutz (WL) test, and/or for directly measuring coincidence with the imaging coordinate system. This test can also be used to determine the mechanical accuracy of gantry and couch angles, and may also be applicable to the comprehensive MV and kV isocenter verification that is used during commissioning and periodic QA. The isocenter verification method is a much more direct and straightforward measurement than the current standard.

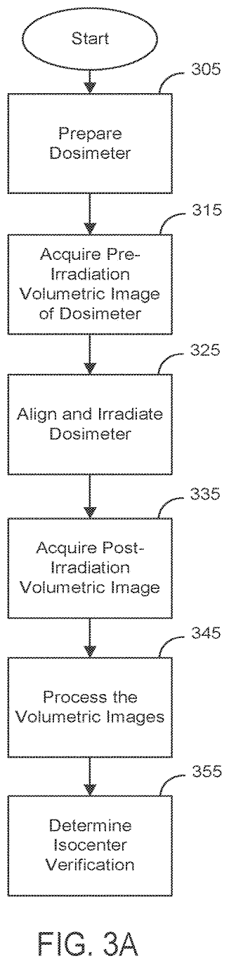

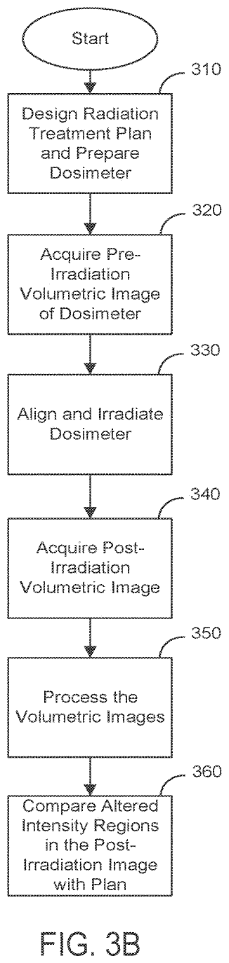

Referring to FIG. 3A, a flowchart of non-limiting example steps for a method of performing isocenter verification of a radiation therapy system is shown. The method includes preparing the dosimeter at step 305. An on-board imaging system may be used to create a pre-irradiation volumetric image of the dosimeter at step 315. As described above, the on-board imaging system may be any appropriate imaging modality, such as a kV Cone-Beam Computed Tomography (CBCT) system. Aligning and irradiating the dosimeter may be performed at step 325. Using the on-board imaging system, a post-irradiation volumetric image of the dose information-containing dosimeter may be acquired at step 335. Image processing of the volumetric images may be performed at step 345. Comparing the spatial location of the altered intensity regions in the post-irradiation volumetric image with a treatment plan may be performed at step 355.

In some configurations, isocenter verification using the dosimeter eliminates potential false positives from user setup error, incorporates evaluation of coincidence with imaging coordinate system, and/or may be applicable to any SRS cone, as well as MLCs for isocentric and multi-target SRS.

In some configurations, an N-isopropylacrylamide (NIPAM) 3D dosimeter for which dose is observed as increased electron density in kV-CBCT may be irradiated at a plurality of couch/gantry combinations, such as eight couch/gantry combinations, which enter the dosimeter at unique orientations. A CBCT may be immediately acquired, radiation profile may be detected per beam, and displacement from imaging isocenter may be quantified.

In one non-limiting example, this test was performed using both 7.5 mm and 4 mm cones, delivering approximately 16 Gy per beam. CBCT settings were 4050 mAs, 80 kVs, smooth filter, 1 mm slice thickness. The 2D displacement of each beam from the imaging isocenter was measured within the planning system. Detectability of the dose profile in the CBCT was quantified as the contrast-to-noise ratio (CNR) of the irradiated high dose regions relative to the surrounding background signal. Setup, irradiation, & readout were carried out within 38 minutes. The 2D vector displacement of each beam from the imaging isocenter was 0.06±0.03 cm (mean±standard deviation), with a range of [0.02 cm 0.11 cm] for the 7.5 mm cone and 0.04±0.01 cm [0.04 cm 0.05 cm] for the 4 mm cone. In comparison, the traditional WL was 0.04±0.01 cm [0.03 cm 0.06 cm]. The CNR of the high dose regions in the CBCT was 3.1 and 1.6 for the 7.5 mm and 4 mm cones, respectively. For the 4 mm cone, the background signal was subtracted from the pre-CBCT, which increased the CNR to 4.0.

A number of treatment delivery parameters may be adjusted to achieve optimal detectability of high dose regions in the dosimeter. These include adjusting the dose distribution (such as selecting for a high dose gradient), radiation beam quality, total dose, dose rate, and the like. High dose falloff and unique geometry per beam for treatment plans may improve the detectability of the high dose regions for measurement of key parameters of the external beam radiotherapy system.

In some configurations, a number of key parameters of the external beam radiotherapy system can be extracted directly from the images. The 3D position and angle of each treatment field may be detected in the post-irradiation images. From this, the geometrical variation of the radiation isocenter as a function of couch and gantry angle can be determined. Any systematic difference between the radiation isocenter and the volumetric imaging coordinate system can be determined by comparing the positions to the coordinate system that is embedded in the volumetric image. The accuracy of gantry and couch angle can also be determined by comparing the angular component of each treatment field with the expected angle. Similar treatment plans can be easily designed and prepared to also evaluate accuracy of collimator angle, jaw positions, and MLC positions.

In one non-limiting example, a single isocenter multifocal VMAT SRS plan was prepared for a dosimeter with 6 targets, each with a 1 cm diameter. A Truebeam STX linear accelerator with 6 MV photons and HD-MLCs was used. The SRS plan utilized 4 non-coplanar VMAT arcs with a 6 MV photon beam. Each target was prescribed a dose of 20 Gy, with the maximum dose being 31.3 Gy.

Referring to FIG. 3B, a flowchart of non-limiting example steps for a method of performing QA for a radiation therapy system is shown. The method includes designing a radiation treatment plan for the QA within the treatment planning system, and preparing the dosimeter at step 310. In some configurations, the QA may be an end-to-end QA. An on-board imaging system may be used to create a pre-irradiation volumetric image of the dosimeter at step 320. As described above, the on-board imaging system may be any appropriate imaging modality, such as a kV Cone-Beam Computed Tomography (CBCT) system. Aligning and irradiating the dosimeter may be performed at step 330. Using the on-board imaging system, a post-irradiation volumetric image of the dose information-containing dosimeter may be acquired at step 340. Image processing of the volumetric images may be performed at step 350. Comparing the spatial location and/or overlap of the altered intensity regions in the post-irradiation volumetric image with the expected high dose regions from a treatment planning system may be performed at step 360.

In some configurations, dosimetric accuracy may also be analyzed in addition to spatial accuracy. The method may also include: pre- and post-irradiation volumetric imaging using a diagnostic imaging system (such as diagnostic CT); defining the relationship between change in intensity and radiation dose via irradiation of a separate dosimeter; defining the relationship between change in intensity and radiation dose via irradiation of a separate portion of the test dosimeter; and specialized image processing of the volumetric images to remove noise and/or extract the dose information.

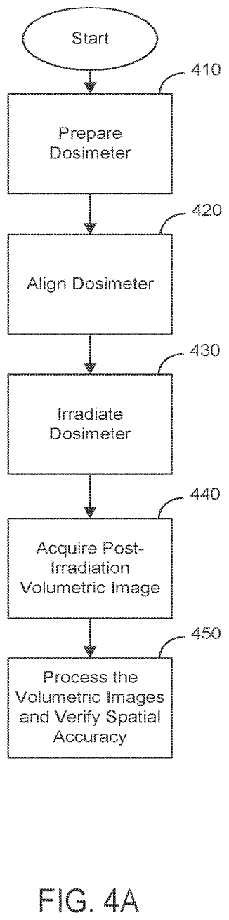

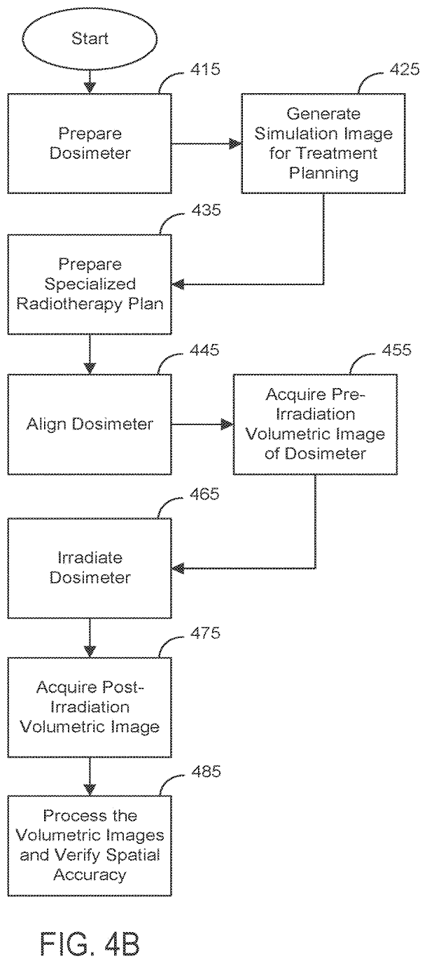

Referring to FIG. 4A, a flow chart of non-limiting example steps for a QA end-to-end test of spatial accuracy are shown. A dosimeter is acquired and/or prepared for irradiation at step 410. Preparation may include mixing the content of the dosimeter, placing fiducial markers on the dosimeter for alignment purposes, and the like. In a non-limiting example, fiducials may be placed on the superior portion of the dosimeter to allow for reproducible setup. The dosimeter may be aligned at the radiation delivery system for irradiation at step 420, and a radiation dose may be delivered at step 430. A post-irradiation volumetric image may be acquired using the on board imager with the dosimeter still aligned at the radiation treatment device at step 440, and the resulting volumetric image may be processed and used to verify the spatial accuracy of the radiation delivery at step 450.

In some configurations, the timing for acquiring the post-irradiated image may be timed to the polymerization of the dosimeter. In one non-limiting example, the polymerization reaction of irradiated NIPAM occurs over several hours where the CT number may change as a function of time post irradiation of NIPAM gel. The time of image acquisition can be optimized so as to maximize the signal received from irradiated regions of the dosimeter.

Referring to FIG. 4B, another flow chart of non-limiting example steps for a QA end-to-end test of spatial accuracy are shown. A dosimeter is acquired and/or prepared as above for irradiation at step 415. A simulation image may be generated at step 425 for treatment planning. The simulation image may be based upon the imaging modality being used in the QA process, such as a simulated CT image. A specialized radiotherapy plan may be prepared at step 435. The dosimeter may be aligned at the radiation delivery system for irradiation at step 445. A pre-irradiation volumetric image of the dosimeter may be acquired at step 455, which may be acquired using the on-board imaging system of the radiotherapy system. A radiation dose may then be delivered at step 465. A post-irradiation volumetric image may be acquired using the on board imager with the dosimeter still aligned at the radiation treatment device at step 475, and the resulting volumetric image may be processed and used to verify the spatial accuracy of the radiation delivery at step 485.

In some configurations, treatment plans may be prepared with commercially available software, such as the Varian External Beam Treatment Planning software version 13.6 (Varian Medical Systems) with the Anisotropic Analytical Algorithm (version 13.6.23).

In some configurations, the pre-irradiation image may be acquired by placing the dosimeter on a treatment table on the radiation therapy system and using an on-board imaging system. The pre-irradiation image may be registered to the planning image and the dosimeter position may be shifted automatically (such as a 6D correction) to eliminate any setup errors, and another image may be acquired to verify the position prior to treatment. The treatment plan may then be delivered as planned, after which a series of image sets may be acquired using appropriate settings. In a non-limiting example of settings that may be used with a CBCT system, the settings may include: 100 kVp, 67 mA, exposure time=7.5 s, with a Ram-Lak convolution kernel applied for image reconstruction.

Image processing may be performed on the images to improve the visibility of the targeted areas. Processing may include averaging multiple images, such as the 3 images discussed above, using morphological operators to remove edges and areas outside the dosimeter, subtracting the median value for columns along the superior-inferior axis, suppressing ring artifacts in each axial slice by subtracting the median value of voxels located at the same distance from the isocenter, equalizing axial slices by subtracting their median value, subtracting a background (pre-irradiation) image, frequency analysis and processing, statistical noise reduction techniques, deep learning based algorithms, and applying filters, such as a low pass filter or utilizing smoothing filters, local and non-local means filters, or non-linear filters. Image processing may greatly improve the detectability of the delivered dose distribution, and the dose distribution could be visualized with an on-board imaging system.

In some configurations, the dose information may be determined from the volumetric images of an on-board imager. The measured spatial location of the dose can be compared to the expected dose distribution and location of each target. In a non-limiting example, the dose distribution may be overlayed with the intensity from the processed images within a clinical Treatment Planning System (TPS).

Parameters of the acquisition technique of the on-board volumetric imaging may be optimized to improve the detectability of the high dose regions in the volumetric image of the dosimeter. Parameters including the kVp, mAs, scan angle, and the use of filters (such as a bow-tie filter), may be optimized to improve the low contrast resolution of the volumetric image. Some techniques applied for other purposes, such as slow gantry rotation for CBCT systems (sometimes used for 4D-CBCT) may improve the image quality. In configurations where the dosimeter shape may be known ahead of time and also kept consistent, the scatter field may also be consistent and therefore may be well modelled and accounted for, provided that the same size dosimeters with same scanning technique are utilized. Alternatively, a scatter grid/measurement hardware could be designed to model and eliminate much of the noise incident on the imager.

Table 1 and 2 depict non-limiting example results for improvement to Contrast to Noise Ratio (CNR) for various acquisition and reconstruction parameters of a kV-CBCT image of a low contrast object within an image quality phantom, which may affect the detectability of the high dose regions of a NIPAM dosimeter.

TABLE 1
Raw contrast and Contrast to Noise Ratio (CNR) from kV-CBCT of
low contrast object in phantom using various reconstruction filters.

| Reconstruction Filter | Raw Contrast | CNR |
|---|---|---|
| Smooth | 26.2 | 9.0 |
| Standard | 28.4 | 6.9 |
| Sharp | 28.0 | 4.3 |
| Automatic (same as Standard) | 25.9 | 6.5 |
| Ultra Sharp | 29.2 | 3.1 |

TABLE 2
Raw contrast and Contrast to Noise Ratio (CNR) from kV-CBCT of
low contrast object in phantom using various kVp settings. The mAs
was adjusted so as to have constant heat loading on the anode.

| kV | mAs | Contrast | CNR |
|---|---|---|---|
| 80 | 6052 | 32.4 | 7.61 |
| 100 | 5085 | 29.3 | 7.73 |
| 125 | 3420 | 30.8 | 9.02 |

In some configurations, choosing optimal parameters for the acquisition of the volumetric image using the on-board imager includes balancing a number of tradeoffs. Attributes of the volumetric image that are affected by the acquisition technique and are advantageous include CNR and spatial resolution. Table 2 shows non-limiting examples of the resulting CNR for various kV settings, when the heat loading on the kV tube is held constant. Higher kV may result in a more optimal CNR, with the ideal setting in the non-limiting example of Table 2 being 125 kV (highest possible for the on-board imaging system that was used). Higher kV resulting in higher CNR may be due to the decreased overall attenuation of the signal through the dosimeter for the higher kV despite the lower raw contrast. Optimization may be possible for other phantom geometries with more or less signal attenuation, which may range from 60-140 kV.

Acquisition time may also be adjusted to optimize the volumetric images. The CNR may be improved without detriment to spatial resolution by acquiring multiple volumetric images and using the average image, but with the cost of increased acquisition time and thus decreased convenience. Acquiring more volumetric images for averaging may also include an increase in imaging dose to the dosimeter, which could increase the background signal in the dosimeter and thus decrease contrast.

The choice of reconstruction filter may also improve the CNR, but may be balanced with a cost of spatial resolution. A smooth reconstruction setting may eliminate a larger amount of high frequency noise, greatly increasing the CNR. The resulting spatial resolution may still allow for accurate visualization of dose gradients for clinical radiotherapy applications.

The mAs parameter may be adjusted. Maximizing the mAs may achieve a higher CNR with fewer acquisitions and thus shorter acquisition time. The mAs may be adjusted up to its maximum setting with potential limitations, such as if the kV tube will overheat prior to completing the scan, if the projection images saturate and cause artifacts in the reconstructed volumetric image, and the like. In such cases, the optimal mAs setting may be the maximum value that does not saturate the projection images or cause overheating for the full CBCT acquisition.

In some configurations, on-board imaging systems allow the option for a partial arc (<360°) and a full arc for CBCT acquisition. CNR may be improved (such as from 1.3 to 5.9) when a full arc (such as 360°) is used. In one non-limiting example, the optimal acquisition settings are: a full arc acquisition, 120 kV, and mAs set to the maximum without saturating the projection image or overheating the tube. The number of acquisitions acquired can then be selected to achieve a desired CNR. The optimal reconstruction may be performed using a smooth reconstruction filter.

In one non-limiting example where a CBCT system is used for the on-board imaging, verification of spatial agreement between the planned and measured treatment dose can be performed without formally defining the relationship between change in Hounsfield Unit and radiation dose (performing a dose calibration). Conversion of Hounsfield unit to dose may not be required with such a method. The method includes using a quasi-dose calibration based solely on the multifocal SRS irradiation (not requiring a second dose calibration irradiation) with only the clinical TPS for analysis. A processed kV-CBCT image may be used, as well as the diagnostic CT images for comparison. The method may include using thresholding tools within the clinical TPS to perform the analysis. Other sophisticated algorithms could also be utilized to identify the volume of interest in the dosimeter, including utilizing morphological operators and gradient analysis, among others.

In addition to the kV-CBCT acquired immediately after irradiation, 5 post irradiation diagnostic CT images were acquired after 36 hours, since the polymerization usually requires on the order of 24 hours to be fully carried out for the dosimeter material used. The CT images were acquired with the following parameters: helical mode, 120 kVp, 530 mA, exposure time 2.341 s, 0.625 slice thickness. NIPAM dosimeters may allow for increased spatial and dosimetric accuracy and precision when imaged with diagnostic multislice CT. In some configurations with CBCT systems, accuracy and precision may be optimized relating to CBCT acquisition by monitoring and adjusting for CBCT housing and anode temperatures, which may affect resultant CT numbers for NIPAM gel. Similar parameters may be taken into consideration in order to establish the resultant accuracy and precision of CBCT NIPAM dosimetry.

In some configurations, within the contouring environment a thresholding tool may be used to create structures that include all voxels with values, such as Hounsfield Units, above a predetermined threshold for increasing threshold levels and for each SRS target. The volumes of these structures may then be compared to the expected volume for the calculated prescription dose volume per target. In addition, the Jaccard index may be calculated for each thresholding structure of each SRS target relative to the TPS prescription dose volume, where the Jaccard index is defined as:

$$J = \frac{\vert {V}_{meas} ⋂ {V}_{TPS} \vert}{\vert {V}_{meas} ⋃ {V}_{TPS} \vert} \qquad ( 1 )$$

where Vₘₑₐₛ is the structure volume from the dosimeter created via image thresholding, and V_{TPS} is the TPS prescription dose volume. This index is the ratio of the intersection and the union of the two volumes, and has a value equal to one when they are the same, but less than one when any size or spatial discrepancy is present. The relative volumes and/or the Jaccard Index may be used to define an appropriate threshold level to compare to a specific dose volume from the treatment plan. The mean spatial position in each axis may also be calculated and compared for each thresholding structure volume and for each prescription dose volume.

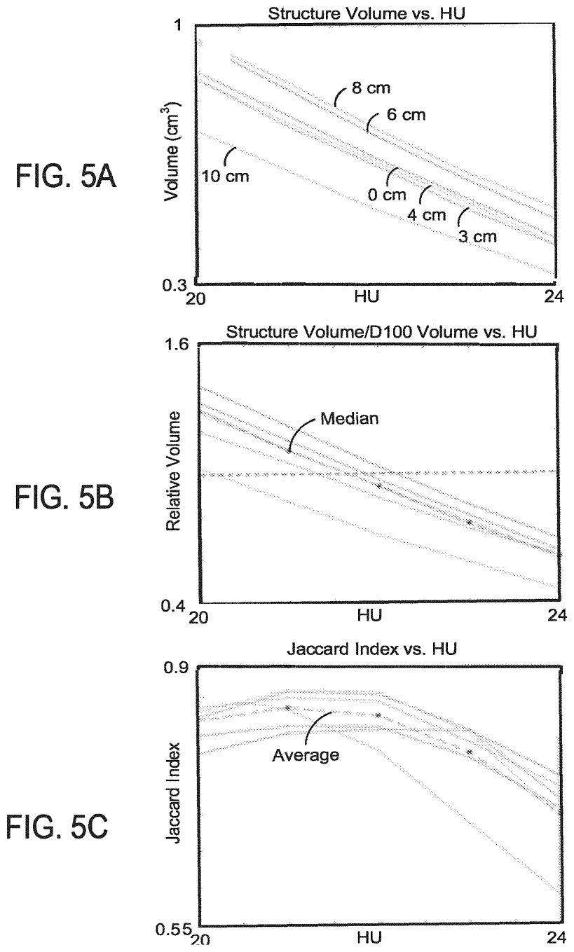

Referring to FIG. 5A, non-limiting example results are shown where the absolute volume of structures created using a thresholding tool in the TPS are graphed as a function of the threshold value for regions of interest surrounding each SRS target. This volume decreases with increasing threshold, as depicted.

Referring to FIG. 5B, non-limiting example results are shown where the volumes shown in FIG. 5A are normalized to the volume of the prescription dose per target from the treatment plan. The volumes depicted are those within the dosimeter that was above a given Hounsfield Unit for each radiosurgery target. The structure created from the threshold will have the same value as the prescription dose (such as 20 Gy) at the point on the curve in where the relative volume has a value of 1 (indicated by the dashed line). For all targets except for the example of 10 cm from isocenter, the value at which this occurred is shown to be tightly grouped within 1 HU of each other. The outlying case (at 10 cm from isocenter) may be indicative of a lower than expected dose for that target, which may also be observed during the analysis with full absolute dose calibration.

Referring to FIG. 5C, non-limiting example results are shown where a similar analysis to that depicted in FIGS. 5A and 5B is used, in which spatial location is also included. The Jaccard Index is shown comparing the structure from thresholding with the prescription dose volume from the treatment plan. The peak value represents the highest amount of overlap between the volumes.

In some configurations, the optimal HU threshold from FIG. 5B may be used to compare the centroid of the prescription isodose cloud from the treatment plan to the centroid of the dose structure from the dosimeter. The difference between the centroids is shown below in Tables 3 and 4 for non-limiting examples where CBCT and diagnostic CT imaging systems were used, respectively.

TABLE 3
non-limiting example centroid comparison for CBCT vs. TPS
CBCT vs. TPS

| | V_{20Gy} Centroid Difference (cm) | | | | |
|---|---|---|---|---|---|
| Target | r-l axis | a-p axis | s-i axis | RMS | Jaccard Index |
| 1 | 0.10 | 0.01 | 0.07 | 0.12 | 0.69 |
| 2 | 0.05 | −0.09 | 0.02 | 0.10 | 0.72 |
| 3 | 0.05 | −0.06 | 0.03 | 0.08 | 0.71 |
| 4 | 0.14 | 0.00 | 0.10 | 0.17 | 0.61 |
| 5 | 0.03 | 0.07 | −0.04 | 0.09 | 0.52 |
| Mean | 0.07 | −0.01 | 0.04 | 0.11 | 0.65 |
| Std dev. | 0.05 | 0.06 | 0.05 | 0.04 | 0.08 |

TABLE 4
non-limiting example centroid comparison for diagnostic
CT vs. TPS
Diagnostic CT vs. TPS

| | V_{20Gy} Centroid Difference (cm) | | | | |
|---|---|---|---|---|---|
| Target | r-l axis | a-p axis | s-i axis | RMS | Jaccard Index |
| 1 | 0.04 | −0.03 | −0.05 | 0.07 | 0.77 |
| 2 | 0.00 | −0.04 | −0.06 | 0.07 | 0.74 |
| 3 | 0.05 | 0.00 | −0.04 | 0.06 | 0.80 |
| 4 | 0.02 | 0.01 | −0.04 | 0.04 | 0.82 |
| 5 | 0.01 | 0.04 | −0.06 | 0.08 | 0.73 |
| Mean | 0.03 | −0.01 | −0.05 | 0.07 | 0.73 |
| Std dev. | 0.02 | 0.04 | 0.01 | 0.01 | 0.10 |

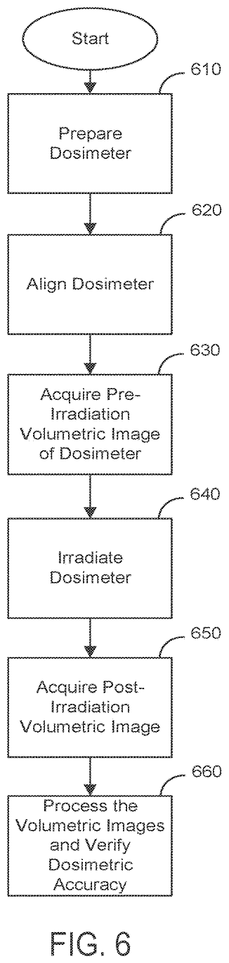

Referring to FIG. 6, a flow chart depicting non-limiting example steps for a method for verification of dosimetric accuracy for multi-target radiosurgery is shown. A dosimeter is acquired and/or prepared for irradiation at step 610. As discussed above, preparation may include mixing the content of the dosimeter, placing fiducial markers on the dosimeter for alignment purposes, and the like. The dosimeter may be aligned at the radiation delivery system for irradiation at step 620. A pre-irradiation volumetric image of the dosimeter may be acquired at step 630. A radiation dose may then be delivered at step 640. A post-irradiation volumetric image may be acquired using the on board imager with the dosimeter still aligned at the radiation treatment device at step 650. The resulting volumetric images may be processed and used to verify the dosimetric accuracy of the radiation delivery at step 660.

In some configurations, the dosimetric accuracy method may be carried out by including specialized pre- and post-irradiation diagnostic volumetric imaging for the dosimetric analysis. Specialized imaging sequences may include those designed to image a dose distribution in the dosimeter. In some configurations, irradiation of a second dosimeter may be included, or a portion of the same dosimeter may be used to define the relationship between radiation dose and change in Hounsfield Units, such as described below. In some configurations, the spatial accuracy method described above may be performed simultaneously with the dosimetric accuracy method.

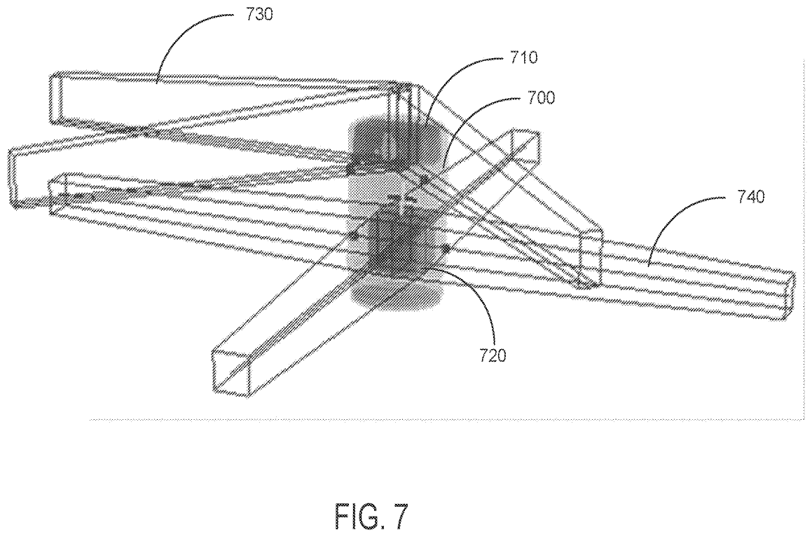

Referring to FIG. 7, a non-limiting example dosimeter 700 is shown that may be used for both dose calibration and to test irradiation, such as by verifying dosimetric accuracy and spatial accuracy as described above. Dosimeter 700 includes dose calibration portion 710, and irradiation test portion 720. Upper portion field irradiation paths 730 and lower portion field irradiation paths 740 demonstrate example radiation beam paths that may be planned for use with the dosimeter 700.

In one non-limiting example where the same dosimeter may be used for both dose calibration and to test irradiation, a treatment plan was prepared for a dosimeter in which the top half included a 3-field irradiation pattern used for the absolute dose calibration, as seen in FIG. 7. The plan utilized three rectangular (3 cm×7 cm) fields at oblique angles, designed so that the high dose area includes a range of dose values to aid in the absolute dose calibration. The prescribed dose was 20 Gy, delivered in a single irradiation with 6 MV photons, with a maximum dose of 27.0 Gy. The lower half of the single dosimeter included a simple 4-field box irradiation, also as shown in FIG. 7, which served as an example of the possibility of including the calibration and test dosimetry in the same dosimeter. The 6 MV photon 4-field box also included a 20 Gy irradiation with open fields of size 5 cm square; the maximum planned dose of the 4 field box was 25.0 Gy.

In some configurations prior to irradiation, images may be acquired of a blank dosimeter (no active ingredient), such as x-ray CT images. A plurality of images may be acquired, such as 5 CTs, as part of the pre-irradiation planning image set. In a non-limiting example where a CT system is used to acquire the pre-irradiation images, appropriate parameters may include: helical mode, 120 kVp, 530 mA, exposure time 2.341 s, 0.625 slice thickness. One skilled in the art will appreciate that other parameters are possible. At the time of irradiation, the dosimeter may be placed on the treatment table and an on-board image may be acquired, such as a CBCT image. The on-board image may be registered to the planning image and the dosimeter position may be shifted automatically (6D correction) to eliminate any setup errors, and another on-board image may be acquired to verify the position prior to treatment. A plurality of post irradiation diagnostic images, such as 5 diagnostic CT images, may be acquired after a specified number of hours, such as 36 hours, since the polymerization usually requires on the order of 24 hours to be fully carried out for some dosimeter materials.

For each dosimeter, an average image set may be created for analysis as the mean of all the CT images. Logical and morphological operators may be used to limit the volume of interest for the analysis to the volume within the dosimeter immediately surrounding the field dose distribution, such as the 3 field dose distribution described above. The dose calibration may be carried out both with and without subtracting the background signal, such as that determined from the blank dosimeter. The conversion of Hounsfield Unit (h) to dose (d) may be performed using the formula:

d=a₁(h−aₛ)^{a}^{₃} (2)

The parameters a₁, a₂, and a₃ may be iteratively optimized to minimize the error fit for all dose voxels above the threshold dose do. This dose calibration may then be used to convert Hounsfield Units to dose for the measured field box, such as the 4-field box described above, and SRS distributions.

In some configurations, a dose calibration method may compare the dose from each pixel in the calculated dose distribution to the CT number measured in the corresponding pixel in the dosimeter image with areas of high-dose gradient removed. A dose calibration curve may be plotted based on these data points using an empirical model described by:

ΔN_{CT}=α+β tan h(γD−φ) (3)

where ΔN_{CT} is the change in CT number; D is the dose; and α, β, γ, and φ are fit parameters. This calibration curve can then be used to convert the CT numbers in the gel image into dose values and create a dose distribution as measured by the dosimeter.

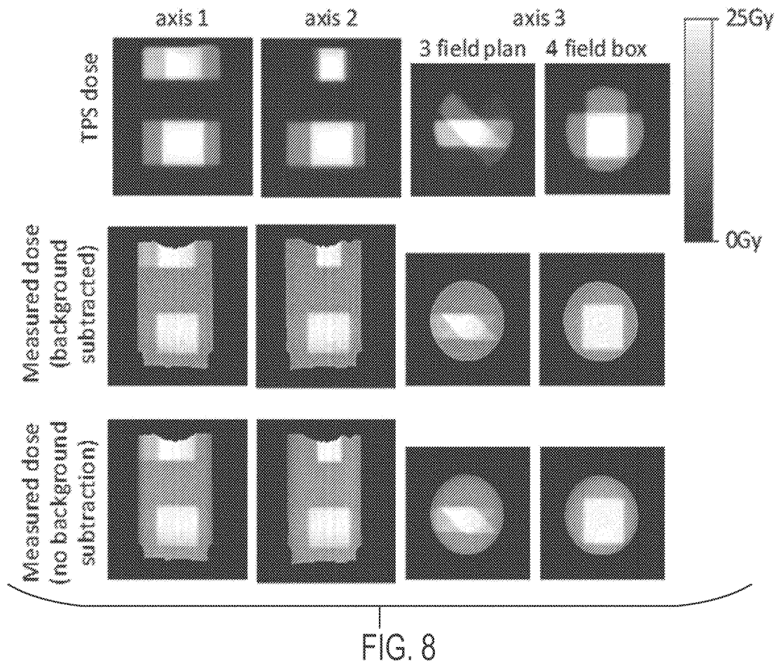

Referring to FIG. 8, non-limiting example results are shown for the planned and measured dose distribution (both when with and without subtracting the background signal) for the method described above where the same dosimeter may be used for both dose calibration and to test irradiation. After fitting the HU values and dose distribution from the 3 field plan, the fit values from equation 1 to convert HU after subtracting the background signal to absolute dose were: a₁=0.850 Gy/HU, a₂=−20.160 HU, and a₃=0.978. When the background was not subtracted, the fit values were a₁=0.027 Gy/HU, a₂=969.477 HU, and a₃=1.6881. For both cases, the threshold dose do below which voxels were excluded from the analysis was 10 Gy. This threshold may be based upon where the dosimeter signal loses proportionality with dose.

One skilled in the art will appreciate that external radiotherapy systems with other mounted kV-volumetric imaging systems, such as systems with CT-on-rails or integrated kV- or MV-CT may be used with the systems and methods of the present disclosure. In instances where the integrated volumetric imaging system is radiographic, gel formulation dosimeters, as well as procedures for image processing and analysis may be applicable. In some configurations, radiotherapy systems with integrated volumetric MR imaging may be used. In this case, a different dosimeter formulation could be utilized, such as a specific formulation that is optimized for T2 signal rather than a formulation that is optimized for change in electron density.

One skilled in the art will also appreciate that the systems and methods of the present disclosure could be performed at other dose levels. Performing the methods at multiple dose levels may aid in defining the relationship between change in Hounsfield unit and planned dose, thus enabling a dose calibration within the clinical treatment planning system. For many clinical treatment planning systems, these analyses may be automated using the scripting and plug-in tools that are available.

The present disclosure has described one or more preferred embodiments, and it should be appreciated that many equivalents, alternatives, variations, and modifications, aside from those expressly stated, are possible and within the scope of the invention.

1. A method for quantifying the output of a radiation therapy system by performing a quality assurance (QA) test, the method comprising:
a) positioning a dosimeter containing a polymer gel material in the radiation therapy system, the polymer gel material having a material property that changes in response to therapeutic radiation, the material property being measurable by an imaging modality;
b) acquiring a pre-irradiation volumetric image of the dosimeter;
c) irradiating the dosimeter with therapeutic radiation using the radiation therapy system to change the material property of the dosimeter and create a region of increased contrast in the polymer gel material of the dosimeter;
d) acquiring a post-irradiation volumetric image of the dosimeter to image the region of increased contrast;
e) generating a QA report based upon the post-irradiation image with the region of increased contrast and the pre-irradiation image.

2. The method of claim 1, wherein acquiring the pre-irradiation volumetric image and the post-irradiation volumetric image includes using an imaging system on-board the radiation therapy system, wherein the on-board imaging system and radiation system share a calibration frame of reference.

3. The method of claim 2, wherein the on-board imaging system includes at least one of an x-ray system, a Cone-Beam Computed Tomography (CBCT) system, a Computed Tomography (CT) system, a 4DCT system, or a magnetic resonance imaging (MRI) system.

4. The method of claim 1, wherein the radiation therapy system includes at least one of an image-guided radiation therapy (“IGRT”) system, an intensity-modulated radiation therapy (“IMRT”) system, an intensity-modulated arc therapy (“IMAT”) system, a volumetric modulated arc therapy (“VMAT”) system, an external beam radiotherapy delivery system, a linear accelerator (LINAC), a proton radiotherapy system, a slice by slice photon radiotherapy system, a non-isocentric photon radiotherapy system, or a isotope based radiotherapy system.

5. The method of claim 1, wherein generating the QA report includes determining at least one of an uncertainty of a radiation isocenter, a coincidence of imaging and radiation coordinate systems, a mechanical accuracy of accelerator geometrical parameters, dosimetric accuracy, or spatial accuracy.

6. The method of claim 5, wherein determining the uncertainty of the radiation isocenter includes irradiating the dosimeter at a plurality of orientations, determining a radiation profile for each region of increased contrast with the post-irradiation volumetric image, and determining displacement from the imaging isocenter for the regions of increased contrast.

7. The method of claim 5, wherein determining spatial accuracy includes generating a treatment plan and comparing a spatial location of the region of increased contrast in the post-irradiation image with the treatment plan.

8. The method of claim 5, wherein determining dosimetric accuracy includes generating a treatment plan and comparing a volume of the region of increased contrast to a volume from the generated treatment plan.

9. The method of claim 8, wherein the dosimetric accuracy is based upon a Jaccard index determined by:
$$J = \frac{\vert {V}_{meas} ⋂ {V}_{TPS} \vert}{\vert {V}_{meas} ⋃ {V}_{TPS} \vert}$$
where Vₘₑₐₛ represents the structure volume from the dosimeter determined from image thresholding, and V_{TPS} represents treatment plan prescription dose volume.

10. The method of claim 5, wherein determining dosimetric accuracy includes determining a conversion of Hounsfield Unit (h) to dose (d) using:
d=a₁(h−aₛ)^{a}^{₃}
where a₁, a₂, and a₃ represent parameters that minimize the error fit for dose voxels above a threshold dose.

11. The method of claim 1, wherein the pre-irradiated volumetric image is subtracted as background from the post-irradiated volumetric image.

12. The method of claim 1, wherein the material property is density and the change in the material property in response to therapeutic radiation is an increase in the density.

13. The method of claim 1, further comprising aligning the dosimeter in the radiation therapy system.

14-26. (canceled)

27. A kit for performing quality assurance (QA) testing of a radiation therapy system, the kit comprising:
i) a dosimeter containing a polymer gel material with a material property that changes in response to therapeutic radiation in a radiation therapy system, wherein the change in the material property is quantifiable as a region of increased contrast in a post-irradiation volumetric image of the dosimeter acquired using an imaging system,
ii) a computer readable medium configured to access the post-irradiation volumetric image of the dosimeter, and
iii) instructions stored on the computer readable medium for identifying the region of increased contrast and generating a QA report based upon the post-irradiation image with the region of increased contrast.

28. (canceled)

29. A dosimeter for use in a radiation therapy system, comprising:
a polymer gel including an oxygen scavenger material and a material with a material property that changes in response to therapeutic radiation in a radiation therapy system, and
wherein the change in the material property is quantifiable using an imaging system.

30. The method of claim 1, wherein the polymer gel material includes at least one of 3-20 wt % of N-isopropylacrylamide (NIPAM), 3-7 wt % of N,N′-methylenebisacrylamide (bis), 2-10 wt % of gelatin, or a combination thereof.

31-34. (canceled)

35. The method of claim 1, wherein the dosimeter further comprises at least one of de-ionized water, methacrylic acid, gelatin, agarose, gellan gum, a layer of mineral oil, or acrylamide.

36. (canceled)

37. The method of claim 1, wherein a ratio of NIPAM to bis is used to determine a sensitivity level.

38. The method of claim 1, wherein 10 mM-100 mM of THPC is used for remote dosimetry.

39-42. (canceled)

43. The method of claim 1, wherein generating the QA report includes determining at least one of an uncertainty of a radiation isocenter; a coincidence of imaging and radiation coordinate systems; a mechanical accuracy of accelerator geometrical parameters; dosimetric accuracy; spatial accuracy.

44-47. (canceled)
